(19) United States
(12) Patent Application Publication
(10) Pub. No.: US 2006/0149320 A1
(43) Pub. Date: Jul. 6, 2006

(54) Implantable cardiac stimulator

(76) Inventors: Sven Hansen, Berlin (DE); Tino Hauser, Berlin (DE)

Correspondence Address: HAHN LOESER & PARKS, LLP, One GOJO Plaza, Suite 300, AKRON, OH, 44311-1076, US

(21) Appl. No.: 10/540,308

(22) PCT Filed: Dec. 17, 2003
(86) PCT No.: PCT/EP03/14429

§ 371 Date: Dec. 12, 2005

(30) Foreign Application Priority Data
Dec. 20, 2002 (DE) 102-61-821.6

(51) Int. Cl. A61N 1/39; A61N 1/362

(52) U.S. Cl. 607/4; 607/9; 607/5

(57) ABSTRACT

An implantable cardiac stimulator, comprises a ventricular detection unit which is adapted to be connected to an intracardiac electrode and to record and detect ventricular events, a ventricular stimulation unit which is adapted to be connected to a ventricular electrode and to produce ventricular stimulation pulses for delivery to the ventricle of a heart, and a control unit connected to the ventricular detection unit and to the ventricular stimulation unit and adapted to actuate the ventricular stimulation unit in a VVI mode in ventricle-inhibited fashion in such a way that a ventricular stimulation pulse is triggered at a moment predetermined by a stimulation rate if it is not inhibited by detection of a natural ventricular contraction by the ventricular detection unit within a predetermined time window. The control unit is adapted to predetermine a stimulation rate, which is higher than intrinsic rate appropriate to the physiological demand.

### BACKGROUND OF THE ART

The invention concerns an implantable cardiac stimulator, in particular a cardiac pacemaker or cardioverter/defibrillator (ICD). The cardiac stimulator includes a ventricular detection unit which is to be connected to an intracardiac electrode and is adapted to register and detect ventricular events. The cardiac stimulator also includes a ventricular stimulation unit which is to be connected to a ventricular electrode and is adapted to produce ventricular stimulation pulses for delivery to the ventricle of a heart. The cardiac stimulator further includes a control unit which is connected to the ventricular detection unit and to the ventricular stimulation unit and is adapted to actuate the ventricular stimulation unit in the VVI mode (ventricle-inhibited) in such a way that a ventricular stimulation pulse is triggered at a moment in time predetermined by a stimulation rate, if it is not inhibited by detection of a natural ventricular contraction by means of the ventricular detection unit within a predetermined time window. The predetermined time window is frequently referred to as the escape interval.

Implantable cardiac pacemakers which are designed for the stimulation of a human heart in a ventricle-inhibited mode are basically known. The various stimulation and sensing modes are generally uniformly identified by a three-letter code of which the first letter denotes the stimulation location (V=ventricle, A=atrium, D=ventricle and atrium), the second letter denotes the sensing location (V=ventricle, A=atrium, D=ventricle and atrium) and the third letter denotes the operating mode (I=inhibited, T=triggered, D=both inhibited and also triggered). Particularly for dual-chamber cardiac pacemakers in the DDD mode, it is also known to effect ventricular stimulation in synchronous relationship with an atrial heart rate which is as natural as possible. If a healthy natural heart rate is not to be found in the atrium, for example in the case of atrial tachycardia or atrial fibrillation, basically atrium-synchronous cardiac pacemakers frequently involve mode switching from atrium-synchronous ventricular stimulation to atrium-asynchronous stimulation in the VVI mode if a detected atrial rate is outside admissible limits.

It is further known for ventricular tachycardias to be treated in the context of a cardioversion therapy by stimulation at a stimulation rate which is above the tachycardia rate. It is possible in that way to break a series of supraventricular and ventricular tachycardias. The aim is in particular to interrupt re-entry circles by an ectopic stimulus formation center being depolarized prematurely by stimulation before a trinsic stimulus can become effective. The stimulation frequency for overdrive stimulation is generally so selected that it is between ten and fifteen pulses per minute higher than the tachycardia to be terminated. The state of the art provides a summary of approaches in terms of how to react to existing tachycardias. One reaction to atrial tachycardia is for example mode switching into the VVI mode and as a reaction to ventricular tachycardia it is possible to provide for triggering overdrive stimulation as a cardioversion therapy.

It is an aspect of the invention to provide a cardiac stimulator with which the occurrence of ventricular tachycardias can be prevented as far as possible from the outset.

### SUMMARY OF THE INVENTION

In accordance with that invention the aspect is attained by an implantable cardiac stimulator of the kind set forth in the opening part of this specification, in which the control unit is adapted to predetermine a stimulation rate which is higher than an intrinsic rate appropriate to the physiological demand. That predetermined stimulation rate is preferably variable and is also referred to hereinafter as the overstimulation rate in order to make it clear that the overstimulation rate is higher than a rate adapted to the physiological demand.

Unlike all known cardiac pacemakers, the cardiac pacemaker according to the invention is thus intended to operate in a kind of overdrive mode constantly and not only when a tachycardia condition prevails. Usually, the term overdrive rate is used to denote a rate which is above a reference rate, namely, in known cardioverters, above a tachycardia rate. For the pacemaker claimed herein, the reference rate for the claimed permanent overdrive stimulation is not a pathological rate but a natural intrinsic rate, which for example, corresponds to a healthy atrial heart rate or which is derived from a per se known manner from parameters or measurement values characterizing the physiological demand of a patient. The terms overstimulation rate or overdrive stimulation rate in the sense used herein are not to be confused with the known overdrive stimulation rate which is used for tachycardia treatment. Overdrive stimulation rate in the conventional sense means a rate which is greater than a tachycardia rate while what is meant here is a rate which is only slightly greater than a healthy, physiologically adequate rate and at any event much less than a tachycardia rate.

The last-mentioned case concerns a rate-adaptive pacemaker in which a stimulation rate for the cardiac pacemaker is so set that the stimulation rate depends on the physiological demand of a patient, thus for example rises with increasing effort, as is also the case with a healthy heart.

In accordance with the concept of permanent overstimulation in an alternative embodiment the control unit of the implantable cardiac pacemaker is adapted to predetermine a fixed stimulation rate of between 70 and 80 stimulation pulses or beat per minute, in particular a rate of about 80 per minute. Such a stimulation rate is between about ten and twenty beats per minute above a natural heart rate of a patient in the rest condition.

In a preferred configuration the control unit of the implantable cardiac pacemaker is adapted to automatically form the overstimulation rate (overdrive stimulation rate) for permanent overstimulation. For that purpose, it is particularly provided that the control unit is adapted to predetermine the overstimulation rate in direct or indirect dependence on transconductions of atrial stimuli by way of an AV node of a heart from the atrium to the ventricle of the heart, in such a way that the number of transconductions or the number of transconducted stimuli within a predetermined time, or in relation to a predetermined number of ventricular events, does not exceed a predetermined degree. The ventricular events can be either stimulated or natural events. The control unit is thus so designed that, by evaluation of the events or other measurement values, in a retrospective period of time, it automatically ascertains a patient-individual, optimum overstimulation base rate. The term overstimulation base rate means herein the overstimulation rate to be provided in the rest condition of the patient. That can be increased in the context of rate adaptation in the case of increased physical effort on the part of the patient.

The choice of the retrospective period of time, in accordance with the above-mentioned predetermined time or the predetermined number of ventricular events, can preferably be variably predetermined.

In order to achieve adaptation of the overstimulation rate, which is as fast as possible, it is preferably provided that the overstimulation rate is already to be increased when only a single transconduction phenomenon or a single transconducted stimulus is detected by the control unit. In that case, the control unit is so designed that it automatically increases the overstimulation rate as soon as transconduction or a transconducted stimulus is detected.

Preferably, the control unit increases the overstimulation rate only for a given period of time and thereafter it decreases it again. That ensures that an excessively high overstimulation rate is not permanently set. On the other hand, the number of possible transconductions is effectively reduced. In an alternative preferred embodiment, the control unit is adapted to increase the stimulation rate as soon as between ten and twenty percent of transconductions or between ten and twenty percent of transconducted stimuli in relation to a total number of ventricular events are detected in the previous period of time. In other words, the control unit sets an increased stimulation rate as soon as the total number of detected ventricular events in a retrospective period of time involves between 10% and 20% of such events which are based on transconductions.

In connection too with the last-mentioned, alternatively preferred embodiment, it is preferably provided that an increase in the overstimulation rate is reversed again after a predetermined time, possibly in steps.

In a further alternative embodiment, the control unit is adapted to set the overstimulation rate in dependence on the number of episodes of ventricular tachycardia within a predetermined period of time or in relation to a predetermined number of ventricular events. An advantage of this configuration over alternative configurations is in particular that tachycardia episodes can be easily detected.

Preferably, the control unit in accordance with the last-mentioned configuration is adapted to increase the variable stimulation rate if the number of episodes of ventricular tachycardia exceeds a predetermined limit value. In a particularly preferred configuration, that predetermined limit value is 5 percent of episode-ventricular tachycardia in relation to the total number of ventricular events in a retrospective observation period of time.

It is preferably also provided in this last-mentioned configuration, that the overstimulation rate is reduced again after an increase. As in all the above-mentioned cases, the gradual reduction in the increased overstimulation rate can take place after a predetermined time in one step or in a plurality of steps after a respective predetermined time. In that case, the time factors can be specified in minutes or hours or however, can also be in the form of a predetermined number of cardiac cycles.

In a particularly preferred variant, the control unit is connected to a sensor with which a measurement value dependent on the physical activity of a patient is to be ascertained. In that case, the control unit is adapted, in dependence on the measurement value, to ascertain a stimulation rate adapted to the physiological demand and to set a suitable overstimulation rate which is above same.

### BRIEF DESCRIPTION OF THE DRAWINGS

The invention will now be described in greater detail by means of an embodiment with reference to the drawings in which:

FIG. 1 shows a perspective view of a dual-chamber cardiac pacemaker with connected and implanted atrial and ventricular electrodes, and

FIG. 2 shows a roughly schematic block diagram of a cardiac pacemaker according to the invention.

### DETAILED DESCRIPTION OF THE INVENTION

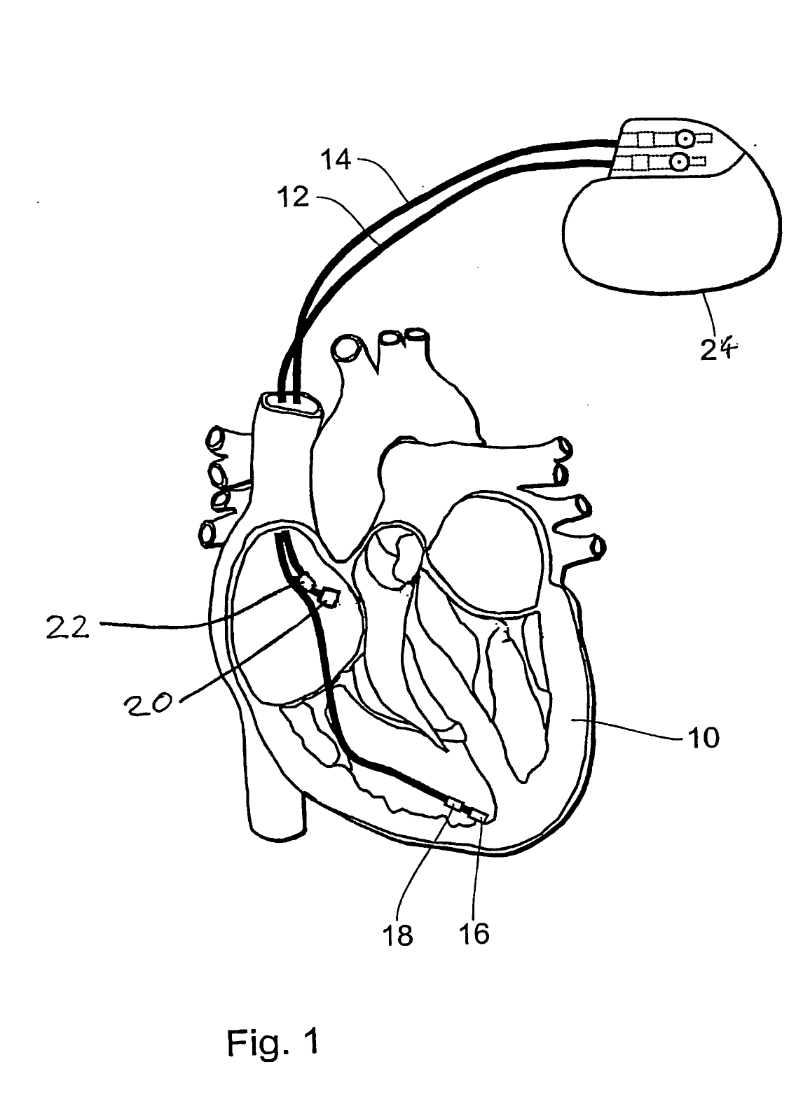

FIG. 1 diagrammatically shows a human heart 10, into the ventricle of which is inserted a ventricular electrode line 12 and into the atrium of which is inserted an atrial electrode line 14. The ventricular electrode line 12 has a ventricular tip electrode 16 and a ventricular ring electrode 18. The atrial electrode line 14 has an atrial tip electrode 20 and an atrial ring electrode 22. The electrode lines 12 and 14 are connected to a dual-chamber cardiac pacemaker 24.

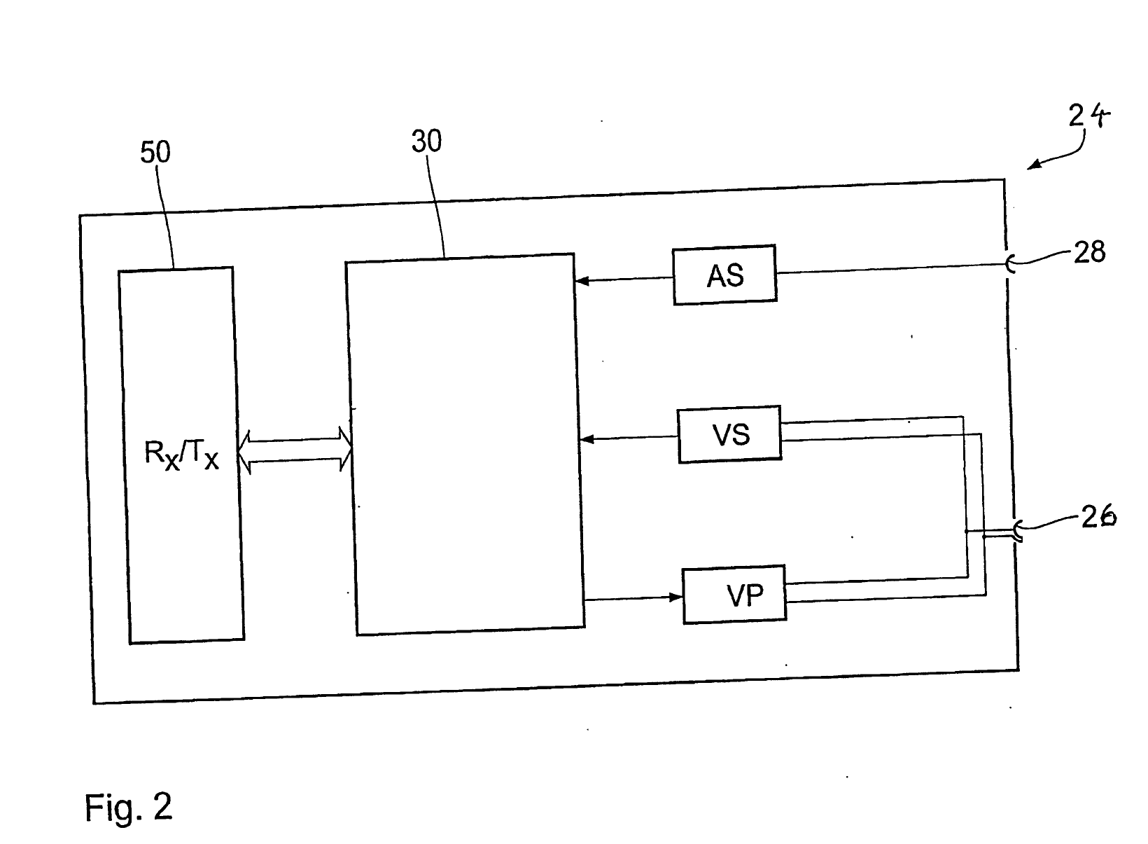

FIG. 2 shows the components of the cardiac pacemaker 24. These are a bipolar electrode line connection 26 for a ventricular electrode line and a unipolar or bipolar electrode line connection 28 for an atrial electrode line. The illustrated simple embodiment of the cardiac pacemaker 24 further has a ventricular detection unit VS and a ventricular stimulation unit VP which are connected by way of the electrode line connection 26 with a ventricular electrode line to the electrode line 12 in FIG. 1. The ventricular detection unit VS serves for recording electrical signals of the heart, for example an intracardiac electrocardiogram. The ventricular stimulation unit VS includes a stimulation pulse generator with associated energy storage devices, charge pump etc as is known from the state of the art, and is suitable for generating and, if required, delivering ventricular stimulation pulses. For triggering ventricular stimulation pulses, the ventricular stimulation unit VS is connected to a control unit 30. The control unit 30 is also connected to the ventricular detection unit VS and to an atrial detection unit AS. The atrial detection unit AS is to be connected by way of the electrode line connection 28 to an atrial electrode line like the electrode line 14.

For communication with external devices, the control unit 30 is provided with a telemetry unit Rx/Tx for the wireless communication, for example, of intracardially recorded electrocardiograms from the cardiac pacemaker 24 to an external device or for the transmission of programming commands from an external device to the cardiac pacemaker 24.

The drawing does not show an atrial stimulation unit which is potentially to be provided, and which enables the cardiac pacemaker 24 also to stimulate the atrium of a heart. Such a dual-chamber cardiac pacemaker in the narrower sense can be operated, for example, in the per se known DDD mode. Means (not further shown) for detecting intracardiac impedance can be used in such a cardiac pacemaker on the one hand for detecting stimulation success (capture detection) immediately after the delivery of a stimulation pulse so that, in the case of stimulation success possibly failing to occur, a back-up pulse can be triggered immediately. In the present case, the per se known means for ascertaining the physiological demand of a patient in the case of physical effort will not be further discussed in greater detail. Such means can be, for example, a blood oxygen sensor or again, an impedance sensor which derives a control signal for the heart rate from the integral of an intracardiac impedance pattern so that the heart rate, in the sense of a closed regulating circuit (closed-loop stimulation), can be adapted to the physiological demand.

What is of interest in the present case is, in particular, a VVI mode in which the cardiac pacemaker 24 can be operated. The stimulation rate, which is to be used as the basis for that mode of operation, can be controlled in the sense of a rate-adaptive cardiac pacemaker in dependence on the physiological demand or it can be a fixedly predetermined base rate. A rate-adaptive cardiac pacemaker 24 however, is preferred. That operation of determining physiological demand, which is required for that purpose, can be effected, for example, as indicated, by means of a blood oxygen sensor or by evaluation of an intracardiac impedance pattern recorded by means of suitable sensors. In that case, the sensors can be the electrodes 16 through 22, including the housing of the cardiac pacemaker 24. Evaluation of the intracardiac impedance pattern is effected in the control unit 30.

In addition, depending on the respective patient, it is possible for a healthy intrinsic heart rate in the atrium of the heart 10 to be detected by means of the atrial detection unit AS.

For the VVI mode of operation of the cardiac pacemaker 24, which is of interest here, firstly a base rate is ascertained on the basis, for example, of an atrial heart rate detected with the atrial detection unit AS, or by calculation based on the physiological demand of the patient. The control unit 30 is then so designed as to form from the variable base rate, a variable stimulation rate as an overstimulation rate, which is for example between 10 and 20 beats per minute higher than the base rate adapted to the physiological demand. The control unit 30 ascertains the precise value of the overstimulation rate on the basis of detected stimulus transconductions from the atrium to the ventricle. Such stimulus transconductions can be ascertained for example by evaluation of the signals recorded in the atrium by means of the atrial detection unit AS, in comparison with corresponding signals detected in the ventricle by means of the ventricular detection unit VS. The overstimulation rate to be ascertained by the control unit 30 is increased if, in a predetermined retrospective period of time (for example 20 cardiac cycles), more than 10% or 20% stimulus transconductions from the atrium to the ventricle are detected. If, conversely, no stimulus transconductions are detected in that retrospective period of time, the overstimulation rate is gradually reduced. Reducing the overstimulation rate increases the probability of transconduction of atrial stimuli to the ventricle. As soon as a predetermined number of transconductions is detected in the predetermined retrospective period of time, the overstimulation rate is increased again. In that way, transconductions of stimuli from the atrium to the ventricle are naturally substantially prevented and thus also, as far as possible, the occurrence of ventricular tachycardias.

1. An implantable cardiac stimulator, in particular a cardiac pacemaker or cardioverter/defibrillator (ICD), comprising a ventricular detection unit (VS) which is to be connected to an intracardiac electrode and is adapted to record and detect ventricular events, and a ventricular stimulation unit (VP) which is to be connected to a ventricular electrode and is adapted to produce ventricular stimulation pulses for delivery to the ventricle of a heart, and a control unit which is connected to the ventricular detection unit (VS) and to the ventricular stimulation unit (VP) and is adapted to actuate the ventricular stimulation unit (VP) in a VVI mode in ventricle-inhibited fashion in such a way that a ventricular stimulation pulse is triggered at a moment in time predetermined by a stimulation rate if it is not inhibited by detection of a natural ventricular contraction by means of the ventricular detection unit (VS) within a predetermined time window, characterized in that the control unit is adapted to predetermine a stimulation rate which is higher than an in particular intrinsic rate appropriate to the physiological demand.

2. A cardiac stimulator as set forth in claim 1 characterized in that the control unit is adapted to predetermine a fixed stimulation rate of between 70 and 90 per minute, preferably about 80 per minute.

3. A cardiac stimulator as set forth in claim 1 characterized in that the control unit is adapted to predetermine a variable stimulation rate in dependence on indirectly or directly detected transconductions of atrial stimuli by way of an AV node of a heart from the atrium to the ventricle of the heart, in such a way that the number of transconductions or the number of transconducted stimuli within a predetermined time or in relation to a predetermined number of ventricular events does not exceed a predetermined degree.

4. A cardiac stimulator as set forth in claim 3 characterized in that the control unit is adapted to increase the variable stimulation rate if the number of transconductions or the number of transconducted stimuli exceeds the predetermined degree.

5. A cardiac stimulator as set forth in claim 4, wherein the predetermined degree is a single transconduction or a single transconducted stimulus.

6. A cardiac stimulator as set forth in claim 4, wherein the predetermined degree is between 10 and 20% of transconductions or between 10 and 20% of transconducted stimuli in relation to a total number of ventricular events.

7. A cardiac stimulator as set forth in claim 6, wherein the control unit is adapted to set a ventricular stimulation rate in dependence on the number of episodes of ventricular tachycardia within a predetermined period of time or in relation to a predetermined number of ventricular events.

8. A cardiac stimulator as set forth in claim 7, wherein the control unit is adapted to increase the variable stimulation rate when the number of episodes of ventricular tachycardia exceeds a predetermined limit value.

9. A cardiac stimulator as set forth in claim 8, wherein the predetermined limit value is 5% of episodes of ventricular tachycardia in relation to the total number of ventricular events.

10. A cardiac stimulator as set forth in claim 1, wherein the control unit is adapted to form an overstimulation rate as a variable stimulation rate from an atrially detected intrinsic base rate or a physiologically adequate base rate ascertained by detection and evaluation of a physiological demand of a patient, in such a way that the overstimulation rate is higher than the base rate by a difference rate.

11. A cardiac stimulator as set forth in claim 10, wherein the control unit is adapted to set the difference rate in dependence on the number of detected stimulus transconductions from the atrium to the ventricle within a predetermined retrospective period of time, in such a way that the control unit increases the difference rate if the number of detected stimulus transconductions exceeds a predetermined degree.

12. A cardiac stimulator as set forth in claim 4, wherein the control unit is adapted to gradually reduce the variable stimulation rate until an increase in the variable stimulation rate occurs again.

13. A cardiac stimulator as set forth in claim 8, wherein the control unit is adapted to gradually reduce the variable stimulation rate until an increase in the variable stimulation rate occurs again.

14. A cardiac stimulator as set forth in claim 10, wherein the control unit is adapted to gradually reduce the variable stimulation rate until an increase in the variable stimulation rate occurs again.

15. A cardiac stimulator as set forth in claim 3 wherein the predetermined degree is a single transconduction or a single transconducted stimulus.

16. A cardiac stimulator as set forth in claim 3 wherein the predetermined degree is between 10 and 20% of transconductions or between 10 and 20% of transconducted stimuli in relation to a total number of ventricular events.

17. A cardiac stimulator as set forth in claim 1, wherein the control unit is adapted to set a ventricular stimulation rate in dependence on the number of episodes of ventricular tachycardia within a predetermined period of time or in relation to a predetermined number of ventricular events.

18. A cardiac stimulator as set forth in claim 2, wherein the control unit is adapted to set a ventricular stimulation rate in dependence on the number of episodes of ventricular tachycardia within a predetermined period of time or in relation to a predetermined number of ventricular events.

19. A cardiac stimulator as set forth in claim 3, wherein the control unit is adapted to set a ventricular stimulation rate in dependence on the number of episodes of ventricular tachycardia within a predetermined period of time or in relation to a predetermined number of ventricular events.

20. A cardiac stimulator as set forth in claim 4, wherein the control unit is adapted to set a ventricular stimulation rate in dependence on the number of episodes of ventricular tachycardia within a predetermined period of time or in relation to a predetermined number of ventricular events.
